(19) United States
(12) Patent Application Publication
(10) Pub. No.: US 2022/0380733 A1
(43) Pub. Date: Dec. 1, 2022

(54) METHOD FOR CULTURING URINE-DERIVED KIDNEY STEM CELLS AND USE THEREOF

(71) Applicant: REGEND THERAPEUTICS CO., LTD., JIANGSU (CN)

(72) Inventors: WEI ZUO, JIANGSU (CN); TING ZHANG, JIANGSU (CN)

(21) Appl. No.: 17/619,591

(22) PCT Filed: Aug. 7, 2020
(86) PCT No.: PCT/CN2020/107771

§ 371 Date: Dec. 16, 2021

(30) Foreign Application Priority Data
Aug. 20, 2019 (CN) 201910771246.1

(51) Int. Cl. C12N 5/071; A61K 35/22; A61K 49/00

(52) U.S. Cl. CPC C12N 5/0687; A61K 35/22; A61K 49/0097; C12N 2502/13; C12N 2500/38; C12N 2500/32; C12N 2501/33; C12N 2501/30; C12N 2501/11

(57) ABSTRACT

Disclosed is a method for culturing urine-derived kidney stem cells, which belongs to the field of cell biology. The method comprises the following steps: isolating cells from the urine, and then culturing the cells with a culture medium of urine-derived kidney stem cells on feeder cells to obtain the urine-derived kidney stem cells, wherein the feeder cells are fibroblasts, and the culture medium of urine-derived kidney stem cells contains 200-300 mL of DMEM medium, 200-300 mL of F12 medium, 20-70 mL of fetal bovine serum, 0.2-2 mM of L-glutamine, 1-14 ng/mL of insulin, 0.1-1 ng/mL of epidermal growth factor, 5-30 μg/mL of adenine, and 2-20 μg/mL of hydrocortisone. By using the method, kidney stem cells with high proliferation capacity and specificity can be obtained and applied, and thus the regenerative outcome of the kidney tissue after injury can be improved.

### TECHNICAL FIELD

The present application relates to the field of cell biology, in particular to a method for culturing urine-derived kidney stem cells and use thereof.

### BACKGROUND

End-stage kidney failure caused by various kidney-related diseases or toxic substances is a major challenge in the medical field. The only effective radical treatment for kidney failure is in-situ kidney transplantation. However, kidney transplantation however is not available for some end-stage kidney failure patients because of factors such as shortage of donor organs, complexity of surgery, high expense, complicated complications and the like. In recent years, cell transplantation, as an alternative treatment strategy of organ transplantation, has drawn more and more attention in the fields of medical science such as blood system diseases, autoimmune diseases, and functional disorders of heart, liver, kidney and other vital organs. Due to its great application potential, much progress has been made in the field of cell transplantation. Cell transplantation has the advantages of abundant cell sources, non- or minimal-invasion in treatment process, and absence of immune rejection and ethical problems that would be caused by allogenic organ transplantations.

For treatments of acute or chronic kidney injury with cell transplantation, the selection of seed cells is of great importance. Currently, seed cells which have application potential in regenerative medicine field include allogeneic totipotent stem cells, induced pluripotent stem cells, adult pluripotent stem cells and adult tissue-specific stem cells, and the like. Wherein adult tissue-specific stem cells can be obtained from autologous tissues, and have the characteristics of committed differentiation into specific tissues and/or organs. In addition, compared to seed cells such as allogeneic totipotent stem cells and induced pluripotent stem cells, adult tissue-specific stem cells are relatively easier to obtain with good histocompatibility, and have very limited tumorigenicity and ethical disputes. As seed cells, they have much more advantages than other cell types.

In this regard, a research article entitled “human urine-derived stem cell transplantation for the treatment of chronic kidney disease in rats” discloses a method of obtaining adult stem cells having characteristics of mesenchymal stem cells from human urine. In this paper, the isolated adult stem cells are transplanted into the renal cortex on both sides of SD rats with chronic kidney disease, and the results showed that cell transplantation could reduce serum creatinine, increase the glomerular filtration rate and improve the kidney function. Yet, there are multiple cell types existing in the human urine, including mesenchymal stem cells and kidney stem cells, etc. The limited number of kidney stem/progenitor cells in urine have made them difficult to obtain. Therefore, conventional cell culture methods, such as the method reported in the above mentioned article, are ineffective to culture the urine-derived kidney stem cells with regenerative capacity.

The obtained cells are mainly mesenchymal stem cells with various biological characteristics of mesenchymal stem cells. However, since mesenchymal stem cells do not have the ability to differentiate into functional kidney cells, they were believed to play a role in immunoregulation by secreting cytokines, and have a temporary survival period in vivo. Hence, the kidney injury structure cannot be repaired. It is highly likely to lead to disease relapse in the clinical applications, thus having certain limitations (ZHAO Yapei, et al. human urine-derived kidney stem cell transplantation treatment on chronic kidney disease in rats, Chinese Journal of Tissue Engineering Research, 2016, 20 (32): 4838-4844).

Thus, it is of great significance to develop a method for screening and isolating kidney stem cells with capacity to repair the injured kidney structure from the urine in the drug screening, physiological pathology research, cell therapy and kidney tissue engineering.

### SUMMARY

Therefore, the present application provides a method for culturing urine-derived kidney stem cells to overcome the defects of prior art that is incapable to isolate and culture cells with kidney regenerative ability, and therefore difficult to repair the injured kidney structure by cell transplantation.

Provided is a method for culturing urine-derived kidney stem cells, comprising isolating stem cells from urine; culturing the stem cells with a culture medium of urine-derived kidney stem cells on feeder cells to obtain the urine-derived kidney stem cells; wherein the feeder cells are fibroblasts, and the culture medium of urine-derived kidney stem cells contains 200-300 mL of DMEM medium, 200-300 mL of F12 medium, 20-70 mL of fetal bovine serum, 0.2-2 mM of L-glutamine, 1-14 ng/mL of insulin, 0.1-1 ng/mL of epidermal growth factor, 5-30 μg/mL of adenine, and 2-20 μg/mL of hydrocortisone.

Furthermore, the feeder cells are derived from embryonic fibroblasts; and wherein the embryonic fibroblasts is selected from established embryonic fibroblasts cell lines or primary cultured embryonic fibroblasts.

Preferably, the feeder cells are mouse embryonic fibroblasts 3T3-J2.

Furthermore, the method for culturing the urine-derived kidney stem cells comprises the following steps:

an isolating step of stem cells, comprising collecting urine, centrifuging, removing the supernatant and collecting the residual liquid, washing the cells in the residual liquid with a washing buffer, centrifuging to obtain a cell pellet, re-suspending the cells with a urine-derived kidney stem cell culture medium to obtain a suspension of cells;
a preparation step of a feeder cell culture system, comprising plating irradiated-growth-arrested feeder cells on bottom of a culture dish to establish a feeder cell culture system; and
a screening and culturing step of kidney stem cells, comprising plating the suspension of cells onto the feeder cells and culturing to obtain urine-derived kidney stem cells.

Furthermore, in the isolating step of stem cells, a volume ratio of urine to the urine-derived kidney stem cell culture medium is (100-200):1.

Furthermore, said preparation step of a feeder cell culture system comprises performing passage culturing of feeder cells in a feeder cell culture medium until the feeder cells reaching a number of 1×10⁸ to 2×10⁸, digesting the cells, centrifuging, removing the supernatant and collecting a cell pellet, re-suspending the cells to obtain a cell suspension with a concentration of 1×10⁶ to 1×10⁷ cells/mL, irradiating with y rays, and then adding matrigel to the culture dish, incubating, removing the matrigel, and adding the y rays-treated feeder cells at a density of 5×10³ to 2×10⁵ cells/cm² and waiting for cell adherence, thereby obtaining a feeder cell culture system.

Furthermore, said screening and culturing step of kidney stem cells comprises: after adherence of the feeder cells, plating the suspension of cells on the feeder cells and culturing for 3-5 days, and replacing the culture medium for the first time, and thereafter repeating the culture medium replacing step at a frequency of once every 2-3 days to perform passage cultures, thereby obtaining the urine-derived kidney stem cells.

Furthermore, the urine is collected from a mid-stream urine sample of a patient with chronic kidney diseases.

Further provided is use of the above urine-derived kidney stem cells in drug screening, physiological pathology research, cell therapy and kidney tissue engineering.

The technical solutions of the present application have the following advantages:

The method for culturing urine-derived kidney stem cells of the application comprises isolating stem cells from urine; culturing the stem cells with a culture medium of urine-derived kidney stem cells on feeder cells to obtain the urine-derived kidney stem cells; wherein the feeder cells are fibroblasts, and the culture medium of urine-derived kidney stem cells contains 200-300 mL of DMEM medium, 200-300 mL of F12 medium, 20-70 mL of fetal bovine serum, 0.2-2 mM of L-glutamine, 1-14 ng/mL of insulin, 0.1-1 ng/mL of epidermal growth factor, 5-30 μg/mL of adenine, and 2-20 μg/ml of hydrocortisone. The use of the feeder cell culture system combined with the urine-derived kidney stem cell culture medium, the kidney stem cells can be gradually screened-in the culture and passage process to obtain a relatively highly purified kidney stem population, and their characteristics including self-renewing capability and differentiation potential can be well maintained. In this culture system, the obtained kidney stem cells can be long-term cultured for more than 10 generations in vitro, with maintaining their clonal growth pattern, proliferation activity, and typical kidney stem cell marker expression.

### BRIEF DESCRIPTION OF THE DRAWINGS

In order to illustrate the embodiments of the present application and the prior art more clearly, the drawings used in the embodiments and the prior art will be briefly described below. Obviously, the drawings attached in the following description only represent some examples of the present application, and those skilled in the field can obtain other drawings based on these drawings without any creative intellectual work.

FIG. 1 is a photograph showing cell morphology of urine-derived kidney stem cells prepared in Example 1 of the present application;

FIG. 2 is a staining image of human urine-derived kidney stem cells of Example 1 of the present application, wherein A showed SOX9; B showed PAX2; C showed DAPI;

FIG. 3 is a photograph showing cell morphology of human urine-derived kidney stem cells after 10 passages in vitro in Example 2 of the present application;

FIG. 4 is a photograph showing the appearance of a transplanted kidney in the kidney injury model under the brightfield of a stereoscope of Experimental Example 5 of the present application;

FIG. 5 is a photograph showing the appearance of a transplanted kidney in the kidney injury model under the green fluorescent channel of a stereoscope of Experimental Example 5 of the present application;

FIG. 6 is a staining image of the urine-derived kidney stem cells in the kidney tissue transplanted with the urine-derived kidney stem cells of Example 5 of the present application, wherein A showed GFP; B showed SLC22A6; C showed DAPI;

FIG. 7 is a staining image of the urine-derived kidney stem cells in the kidney tissue transplanted with the urine-derived kidney stem cells of Example 5 of the present application, wherein A showed GFP; B showed UMOD; C showed DAPI;

FIG. 8 is a staining image of the urine-derived kidney stem cells in the kidney tissue transplanted with the urine-derived kidney stem cells of Example 5 of the present application, wherein A showed GFP; B showed SYNPO; C showed DAPI;

FIG. 9 is a photograph showing cell morphology of the urine-derived stem cells prepared in Comparative Example 1 of the present application.

### DETAILED DESCRIPTION

The following examples are provided for a better understanding of the application, and are not limited to the best mode, or intended to limit the scope of the application. Any product that is the same or similar to the present application, either inspired by the present application or in combination with other prior art features, falls within the protection scope of the present application.

Experimental steps or conditions not noted in the examples can be carried out according to the operation or condition of the conventional experimental steps described in the literature. The used reagents or instruments, for which the manufacturers are not noted, are all conventional reagent products which can be commercially available.

### Example 1 Isolation, Culture, and Identification of Urine-Derived Kidney Stem Cells

Provided is a method for culturing urine-derived kidney stem cells, comprising the following steps:

(1) Isolating step of stem cells: 3 centrifuge tubes are taken, and an antibiotic cocktail comprising 500 μL of penicillin-streptomycin solution (100×) and 50 μL of 2.5 mg/mL amphotericin solution is added into each centrifuge tube. 150 mL of urine collected from a patient with chronic kidney disease is equally divided into three parts, and each part is added into each of the above 3 centrifuge tubes. The tubes are centrifuged at 420 g for 10 minutes, the supernatant is removed and about 2 mL of residual urine at the bottom of the centrifuge tube is retained. The residual urine in all the centrifuge tubes are pooled together into one centrifuge tube, and a wash buffer is added to reach 50 mL. Then the cell pellet at the bottom is resuspended by pipetting up and down, and centrifuged again at 380 g for 15 minutes, followed by removing the supernatant and adding a wash buffer. The process is repeated for 3 times, and at the last centrifuging, supernatant is fully removed and only the cell pellet at the bottom is retained. 1 mL of kidney stem cell culture medium is added to resuspend the cells by pipetting up and down to obtain a suspension of cells.

The wash buffer should be freshly prepared prior to use, and its formulation comprises a base culture medium and additive components, wherein the base culture medium is F12 medium, and the additive components are: 6% (v/v) of fetal bovine serum, 1% (v/v) of 100×L-glutamine solution, 1% (v/v) of 100× penicillin-streptomycin solution, 2.5 μg/mL of amphotericin, and 100 μg/mL of gentamicin. The kidney stem cell culture medium contains 225 mL of DMEM medium, 225 mL of F12 medium, 50 mL of fetal bovine serum, 1.2 mM of L-glutamine, 5 ng/mL of insulin, 0.5 ng/mL of epidermal growth factor, 30 μg/mL of adenine, and 10 μg/mL of hydrocortisone.

(2) Preparation step of a culture dish pre-coated with feeder cells:

Mouse embryonic fibroblasts 3T3-J2 cells are selected as feeder cells. A feeder cell culture medium is added to perform passage culture until the 3T3-J2 cells reach a number of 1×10⁸. The cultured cells then are digested, centrifuged at 350 g for 10 minutes, and supernatant is discarded and a cell pellet is collected. 30 ml of the feeder cell culture medium is added to resuspend the cell pellet to a concentration of 1×10⁶ cells/30 mL. The suspension is pipetted up and down to mix well, then irradiated with y rays at an irradiation dose of 60 Gy/time, thus obtaining a culture dish having coated with the feeder cells. Then 400 μL of 20% (v/v) matrigel is added into a 12-well plate, and the plate is incubated at 37° C. for 30 minutes. Then the matrigel is removed, and the feeder cells are added at a density of 5×10³ cells/cm² for the feeder cells to cover the whole bottom of the culture plate. The cells are cultured for 6 hours and are ready for use after adherence. The feeder cell culture medium is a DMEM medium supplemented with 10 wt % of FBS, 1 wt % of penicillin-streptomycin solution and 1 wt % of L-glutamine solution.

(3) Screening and culturing step of kidney stem cells: The suspension of stem cells prepared in the step (1) is plated onto the feeder cells, and cultured at 37° C. in a 5% CO₂ incubator. After 3 days, the culture medium is replaced with fresh culture medium for the first time, and then the culture medium replacing is repeated at a frequency of once every 2 days. If cell clones emerge within 14 days, the kidney stem cell isolation and culture is a successful one. The culture is continued in the feeder cell culture system, producing urine-derived kidney stem cells.

The morphology of urine-derived kidney stem cells is observed and recorded with an inverted phase contrast microscopy, and the results are shown in FIG. 1. It is shown that the urine-derived kidney stem cells grow as clones, and the clones have a clear boundary, and cells in the clones are uniform.

P3 (passage number: 3) Urine-derived kidney stem cells at passage 3 (P3) are taken for immunofluorescence staining to identify the kidney stem cell markers SOX9 and PAX2. Specific steps are as follows: When the stem cell clone grows to a size of 20 cells, the cells are fixed using 4% paraformaldehyde for 10 minutes. Then the cells are washed with PBS for 5 minutes, 3 times to remove the residual paraformaldehyde, followed by permeabilization treatment with 2.5% Triton X-100 for 5 minutes. The cells then are washed 3 times with PBS, 5 minutes each time, followed by blocking treatment for 30 minutes with a PBS solution which contains 7% (v/v) donkey serum. After blocking, the donkey serum-PBS solution is removed, and a PBS solution containing primary antibodies (a rabbit-derived anti-SOX9 antibody and a mouse-derived anti-PAX2 antibody) is added thereto to perform incubating overnight at 4° C. The PBS solution containing the primary antibodies is removed, and the cells are washed 3 times with PBS, 10 minutes for each time. A PBS solution containing secondary antibodies (a donkey-anti-rabbit secondary antibody labeled with 594 fluorophore, and a donkey-anti-mouse secondary antibody labeled with 488 fluorophore) is added to perform incubating at room temperature for 2 hours. The PBS solution containing the secondary antibodies is removed, and a 0.1% DAPI solution is added to perform nuclear staining, followed by incubating for 10 minutes. The cells are washed 3 times with PBS, 20 minutes for each time, then mounted and observed under a microscope.

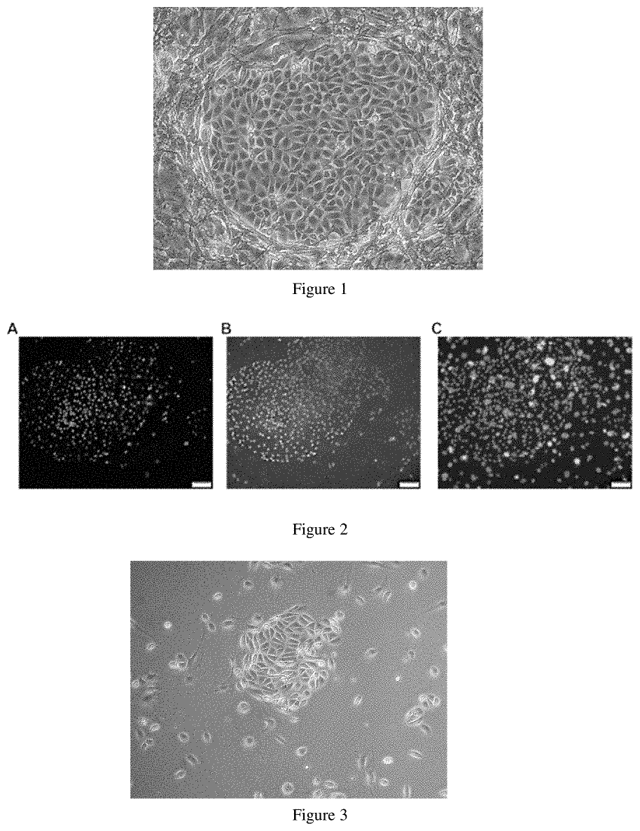

The results are shown in FIGS. 2A-C. Red fluorescence, green fluorescence, and blue fluorescence are observed, respectively. It is indicated that the cells in the present example express kidney stem cell markers SOX9 and PAX2, and the kidney stem cells can be successfully screened and cultured by the method of the present example.

### Example 2 Method for Culturing Urine-Derived Kidney Stem Cells

Provided is a method for culturing urine-derived kidney stem cells, comprising the following steps:

(1) Isolating step of stem cells: 3 centrifuge tubes are taken, and an antibiotic cocktail comprising 500 μl of penicillin-streptomycin solution (100×) and 50 μl of 2.5 mg/ml amphotericin solution is added into each centrifuge tube. 150 mL of urine collected from a patient with chronic kidney disease is equally divided into three equal parts, and each part is added into each of the above 3 centrifuge tubes. The tubes are centrifuged at 380 g for 15 minutes, the supernatant is removed, and about 1 mL of residual urine at the bottom of the centrifuge tube is retained. The residual urine in all the centrifuge tubes are pooled together into one centrifuge tube, and a wash buffer is added to reach 50 ml. Then the cell pellet at the bottom is resuspended by pipetting up and down, and centrifuged again at 420 g for 10 minutes, followed by removing the supernatant and adding a wash buffer. The process is repeated for 2-3 times, and at the last centrifuging, supernatant is fully removed and only the cell pellet at the bottom is retained. 1 mL of kidney stem cell culture medium is added to resuspend the cells by pipetting up and down to obtain a suspension of stem cells.

The wash buffer should be freshly prepared prior to use, and its formulation comprises a base culture medium and additive components, wherein the base culture medium is F12 medium, and the additive components are: 4% (v/v) of fetal bovine serum, 1% (v/v) of 100× L-glutamine solution, 1% (v/v) of 100× penicillin-streptomycin solution, 2.5 μg/mL of amphotericin and 100 μg/mL of gentamicin. The kidney stem cell culture medium contains 225 mL of DMEM medium, 225 mL of F12 medium, 50 mL of fetal bovine serum, 1.0 mM of L-glutamine, 7 ng/mL of insulin, 0.5 ng/mL of epidermal growth factor, 10 μg/mL of adenine, and 10 μg/mL of hydrocortisone.

(2) Preparation step of a culture dish pre-coated with feeder cells:

Mouse embryonic fibroblasts 3T3-J2 cells are selected as feeder cells. A feeder cell culture medium is added to perform passage culture until the 3T3-J2 cells reach a number of 2×10⁸. The cultured cells then are digested, centrifuged at 350 g for 5 minutes, and supernatant is discarded and a cell pellet is collected. 30 ml of the feeder cell culture medium is added to resuspend the cell pellet to a concentration of 1×10⁷ cells/30 mL. The suspension is pipetted up and down to mix well, then irradiated with y rays at an irradiation dose of 60 Gy/time, thus obtaining growth-arrested feeder cells. Then 600 μL of 20% (v/v) matrigel is added into a 12-well plate, and the plate is incubated at 37° C. for 15 minutes. Then the matrigel is removed, and the feeder cells are added at a density of 2×10⁵ cells/cm² for the feeder cells to cover the whole bottom of the culture plate. The cells are cultured for at least 6 hours and are ready for use after adherence. The feeder cell culture medium is a DMEM medium supplemented with 10 wt % of FBS, 1 wt % of penicillin-streptomycin solution and 1 wt % of L-glutamine solution.

(3) Screening and culturing step of kidney stem cells: The suspension of stem cells prepared in the step (1) is plated onto the feeder cells, and cultured at 37° C. in a 5% CO₂ incubator. After 4 days, the culture medium is replaced with fresh culture medium for the first time, and then the culture medium replacing is repeated at a frequency of once every 2 days. If cell clones emerge within 15 days, the kidney stem cell isolation and culture is a successful one. The culture is continued in the feeder cell culture system, producing urine-derived kidney stem cells.
(4) When the stem cells grow to cover 70-90% of the surface area of the culture dish, the supernatant is removed, and the cells are washed once with PBS, and digested with 0.25% Trypsin-EDTA for 4 minutes. After the digestion is terminated, all cell suspension is collected, and centrifuged at 1100 rpm for 3 minutes. The supernatant is removed, and the cells are resuspended with a kidney stem cell culture medium, plated onto a culture dish pre-coated with feeder cells, and cultured at 37° C., 5% CO₂ for next generation culture.
(5) According to the step (4), the cells cultured for many generations and still maintain the characteristics of the kidney stem cells. As shown in FIG. 3, after 10 passages in vitro, the kidney stem cells can still grow as clones and maintain high proliferation activity.

### Example 3 Construction of Kidney Injury Model and Transplantation of Human Urine-Derived Kidney Stem Cells into Mouse Kidney Injury Model

Provided is a method for constructing the kidney injury model and transplantation of human urine-derived kidney stem cells to kidney injury model, comprising the following steps:

(1) 6-8 week-old Non-Obese Diabetes/Severe Combined Immunodeficiency Disease (NOD/SCID) mice are anesthetized by injecting intraperitoneally with 4% chloral hydrate. The mice are placed in a prone position, and a 1 cm cut is made at the left side of the lumbar spine to expose the left kidney. The left renal artery is temporarily closed with a vascular clip to prevent massive blood loss during nephrectomy process. A longitudinal cutting is made along the epitaxial convex side of the kidney, and kidney papilla tissue is excavated with microforceps and scalpels when the kidney is cut in half. The cut is then sealed with a medical adhesive glue, and the mice are hold still for 30 s for the bonding of the cut.
(2) Urine-derived kidney stem cells prepared according to the method disclosed in Example 1 are resuspended with the urine-derived kidney stem cell culture medium to a density of 333333 cells/μL. Then the suspension of urine-derived kidney stem cells is injected with a syringe to the injury site of the left kidney at a dose of 30 μL per mouse. If no obvious liquid leakage is observed, the muscle layer and the skin are sutured, and the abdominal cavity is closed, so that transplantation of the human urine-derived kidney stem cells to the kidney injury mouse is completed.

### Example 4 Green Fluorescent Protein (GFP) Labeling of Human Urine-Derived Kidney Stem Cells

Provided is a method for labeling human urine-derived kidney stem cells, comprising the following steps:

(1) 50000 urine-derived kidney stem cells prepared according to the method disclosed in Example 1 are taken and plated into one well of a 6-well cell culture plate. GFP-expressing lentivirus are added to the cell culture together with 10 μg/mL polybrene according to MOI value of 15. After incubating for 16 hours in a cell culture incubator, the cell culture supernatant is removed, and the cells are washed twice with PBS. Then the kidney stem cell culture medium is added to perform culture.
(2) After 24-48 hours, the cells are observed under a fluorescence microscope. If the cells express GFP, they will be continued to culture and amplify. The obtained cells can be used for transplantation into animal models.

### Example 5 Construction of Kidney Injury Model and Transplantation of Human Urine-Derived Kidney Stem Cells to Kidney Injury Mouse Model

Provided is a method for constructing a kidney injury model, comprising the following steps:

(1) 6-8 week-old Non-Obese Diabetes/Severe Combined Immunodeficiency Disease (NOD/SCID) mice are anesthetized by injecting intraperitoneally with 4% chloral hydrate. The mice are placed in a prone position, and a 1 cm cut is made at the left side of the lumbar spine to expose the left kidney. The left renal artery is temporarily closed with a vascular clip to prevent massive blood loss during nephrectomy process. A longitudinal cutting is made along the epitaxial convex side of the kidney, and kidney papilla tissue is excavated with microforceps and scalpels when the kidney is cut in half. The cut is then sealed with a medical adhesive glue, and the mice are hold still for 30 s for the bonding of the cut.
(2) GFP-expressing urine-derived kidney stem cells prepared according to the method disclosed in Example 4 are resuspended with the urine-derived kidney stem cell culture medium to a density of 25000 cells/μL. Then the suspension of urine-derived kidney stem cells is injected with a syringe to the injury site of the left kidney at a dose of 30 μL per mouse. If no obvious liquid leakage is observed, the muscle layer and the skin are sutured, and the abdominal cavity is closed, so that transplantation of the human urine-derived kidney stem cells to the kidney injury mouse is completed.

### Example 6 Detection of Transplantation of Human Urine-Derived Kidney Stem Cells into Kidney Injury Mouse

Provided is a method for detecting transplantation of human urine-derived kidney stem cells, comprising the following steps:

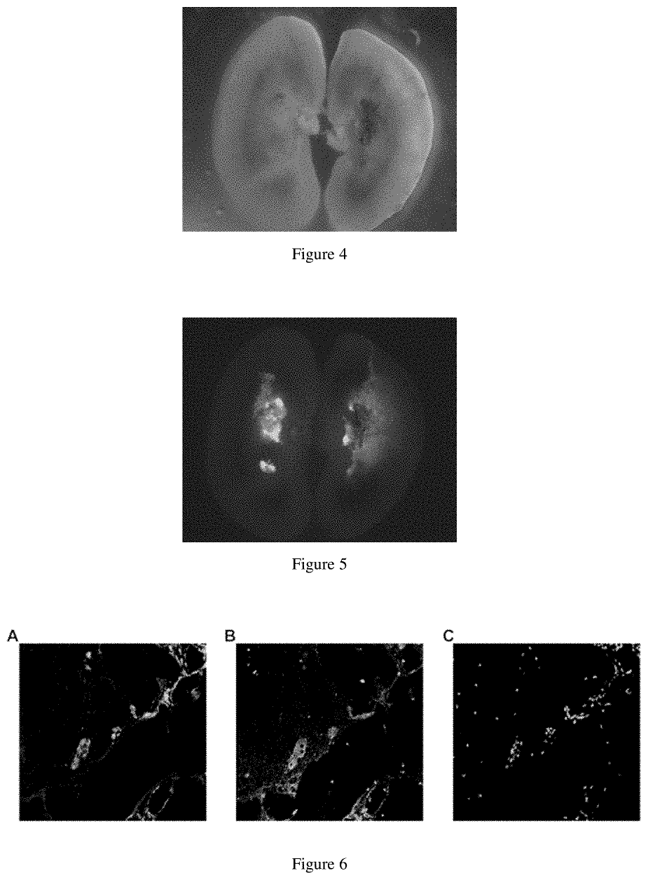

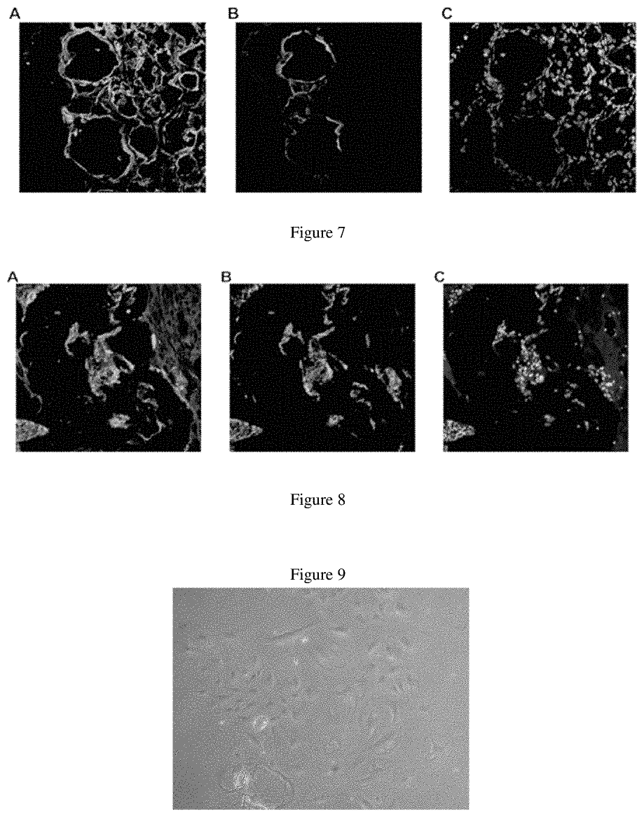

(1) Pathologic identification: The kidney injury mouse after transplantation in Example 5 is anaesthetized with ether and sacrificed by cervical dislocation at 14 days post-transplantation of human urine-derived kidney stem cells. The kidney received transplantation is harvested and examined under a stereomicroscope for fluorescence signals. The results are shown in FIGS. 4 and 5.

FIG. 4 shows a bright-field microscopic image of the kidney after transplantation. FIG. 5 is a fluorescent image under the same field of view, where green fluorescent signals can be clearly observed. It is indicated that the urine-derived kidney stem cells can integrate into the kidney tissue after transplantation into the kidney injury mouse.

(2) Identification of kidney section: The kidney received transplantation is fixed with 3.7% formaldehyde, embedded into O.C.T for overnight at −80° C., and sectioned at a thickness of 8 μm. Corresponding primary antibody (GFP and SLC22A6) or (GFP and UMOD) or (GFP and SYNO) is added, respectively, and then the sections are examined to check fluorescence signals.

FIGS. 6-A, 7-A, and 8-A show that, cells expressing green fluorescent protein are detected in the section, illustrating that human urine-derived kidney stem cells have successfully integrated into the damaged kidneys. At the same time, some of the green-fluorescent-protein-expressing cells exhibit a tubular morphology, and express SLC22A6, UMOD, and SYNOPO, which are key markers of the renal tubules epithelial tissue, indicating that human urine-derived kidney stem cells integrate into the damaged kidney and then differentiate into mature epithelial tissue, generating new kidney tubule tissues. The results show that the urine-derived kidney stem cells prepared by the present method have good kidney regenerative potential.

### Comparative Example 1 Conventional Method for Culturing Urine-Derived Stem Cells

Provided is a method for culturing urine-derived stem cells, comprising the following steps:

(1) Isolation step of stem cells: 3 centrifuge tubes are taken, and an antibiotic cocktail comprising 500 μL of penicillin-streptomycin solution (100×) and 50 μL of 2.5 mg/mL amphotericin solution is added into each centrifuge tube. 150 mL of urine collected from a patient with chronic kidney disease is equally divided into three parts, and each part is added into each of the above 3 centrifuge tubes. The tubes are centrifuged at 420 g for 10 minutes, supernatant is removed and about 2 ml of residual urine at the bottom is retained. The residual urine in all the centrifuge tubes are pooled together into one centrifuge tube, and a wash buffer is added to reach 50 mL. Then the cell pellet is resuspended by pipetting up and down, and centrifuged again at 380 g for 15 minutes, followed by removing the supernatant and adding a wash buffer. The process is repeated for 3 times, and at the last centrifuging, the supernatant is fully removed and only the cell pellet at the bottom is retained. 1 mL of kidney stem cell culture medium is added to resuspend the cells by pipetting up and down to obtain a cell suspension. The cell suspension is directly plated onto a culture plate and cultured without feeder cells.

The wash buffer should be freshly prepared prior to use, and its component comprises a base culture medium and additive components, wherein the base culture medium is a F12 medium, and the additive components are: 6% (v/v) of fetal bovine serum, 1% (v/v) of 100× L-glutamine solution, 1% (v/v) of 100× penicillin-streptomycin solution, 2.5 μg/mL of amphotericin and 100 μg/mL of gentamicin. The kidney stem cell culture medium contains 225 mL of DMEM medium, 225 mL of F12 medium, 50 mL of fetal bovine serum, 1.2 mM of L-glutamine, 5 ng/mL of insulin, 0.5 ng/mL of epidermal growth factor, 30 μg/mL of adenine, and 10 μg/mL of hydrocortisone.

After culturing for 11 days, the morphology of the obtained cells is observed and recorded with an inverted phase contrast microscopy, and the results are shown in FIG. 9. It is shown that the cells in the field of view have morphology significantly different from the morphology of the urine-derived kidney stem cells obtained in Example 1, and the cells do not grow as clones and have a larger size, a poor uniformity, and a slow proliferation rate.

Apparently, the aforementioned embodiments are merely examples illustrated for clearly describing the present invention, rather than limiting the implementation ways thereof. For those skilled in the art, various changes and modifications in other different forms can be made on the basis of the aforementioned description. It is unnecessary and impossible to exhaustively list all the implementation ways herein. However, any obvious changes or modifications derived from the aforementioned description are intended to be embraced within the protection scope of the present invention.

1. A method for culturing urine-derived kidney stem cells, comprising
isolating stem cells from urine;
culturing the stem cells with a culture medium for urine-derived kidney stem cells on feeder cells to obtain the urine-derived kidney stem cells;
wherein the feeder cells are fibroblasts, and the culture medium of urine-derived kidney stem cells contains 200-300 mL of DMEM medium, 200-300 mL of F12 medium, 20-70 mL of fetal bovine serum, 0.2-2 mM of L-glutamine, 1-14 ng/mL of insulin, 0.1-1 ng/mL of epidermal growth factor, 5-30 μg/mL of adenine, and 2-20 μg/mL of hydrocortisone.

2. The method for culturing urine-derived kidney stem cells according to claim 1, wherein the feeder cells are derived from embryonic fibroblasts; and wherein the embryonic fibroblasts are selected from established embryonic fibroblast cell lines and primary cultured embryonic fibroblasts.

3. The method for culturing urine-derived kidney stem cells according to claim 1, wherein the feeder cells are mouse embryonic fibroblasts 3T3-J2.

4. The method for culturing the urine-derived kidney stem cells according to claim 1, comprising the following steps:
an isolating step of stem cells, comprising collecting urine, centrifuging, removing supernatant and collecting residual liquid, washing the cells in the residual liquid with a washing buffer, centrifuging to obtain a cell pellet, and re-suspending the cells with a urine-derived kidney stem culture medium to obtain a suspension of cells;
a preparation step of a culture dish pre-coated with feeder cells, comprising plating irradiated-growth-arrested feeder cells on bottom of a culture dish, followed by placing the culture dish in a cell culture incubator overnight or until the next day; and
a screening and culturing step of kidney stem cells, comprising plating the suspension of cells onto the feeder cells and performing culturing to obtain the urine-derived kidney stem cells.

5. The method for culturing urine-derived kidney stem cells according to claim 4, wherein in the isolating step of stem cells, a volume ratio of urine to the culture medium of urine-derived kidney stem cell is (100-200): 1.

6. The method for culturing urine-derived kidney stem cells according to claim 4, wherein the preparation step of a culture dish pre-coated with feeder cells comprises performing passage culturing of feeder cells in a feeder cell culture medium until the feeder cells reaching a number of 1×10⁸ to 2×10⁸, digesting the cells, centrifuging, removing the supernatant, collecting the cell pellet, and re-suspending the cells to obtain a cell suspension with a concentration of 1×10⁶ to 1×10⁷ cells/mL, irradiating with y rays, and then adding matrigel to the culture dish, incubating, removing the matrigel, and adding the feeder cells at a density of 5×10³ to 2×10⁵ cells/cm² and waiting for cell adherence, thereby obtaining a culture dish coated with the feeder cells.

7. The method for culturing the urine-derived kidney stem cells according to claim 4, wherein said screening and culturing step of kidney stem cells comprises: after adherence of the feeder cells, plating the suspension of cells on the feeder cells and culturing for 3-5 days, and replacing the culture medium for the first time, and thereafter repeating the culture medium replacing step at a frequency of once every 2-3 days, and perform passage cultures, thereby obtaining the urine-derived kidney stem cells.

8. The method for culturing urine-derived kidney stem cells according to claim 1, wherein the urine is collected from a mid-stream urine sample of a patient with chronic kidney diseases.

9. (canceled)

10. A method for treating kidney injury, comprising transplanting the urine-derived kidney stem cells prepared by the method of claim 1 into the kidney injury.

11. The method according to claim 10, comprising preparing the urine-derived kidney stem cells into a suspension with a density of 25000 cells/μL to 333333 cells/μL, and injecting the suspension to a site of the kidney injury.

12. The method according to claim 10, comprising labeling the urine-derived kidney stem cells with green fluorescent protein in prior to the transplanting.
